(19) United States
(12) Patent Application Publication
(10) Pub. No.: US 2020/0060662 A1
(43) Pub. Date: Feb. 27, 2020

(54) METHODS AND DEVICES FOR GASEOUS SAMPLE COLLECTION AND STORAGE

(71) Applicants: Ghassan S. Kassab, La Jolla, CA (US); Klaus Ley, La Jolla, CA (US)

(72) Inventors: Ghassan S. Kassab, La Jolla, CA (US); Klaus Ley, La Jolla, CA (US)

(21) Appl. No.: 16/551,623

(22) Filed: Aug. 26, 2019

Related U.S. Application Data
(60) Provisional application No. 62/722,851, filed on Aug. 25, 2018.

(51) Int. Cl. A61B 10/00

(52) U.S. Cl. CPC A61B 10/0096; A61B 2010/0083

(57) ABSTRACT

Methods and devices for gaseous sample collection and storage. In an exemplary embodiment of a device for collecting and storing a gaseous sample of the present disclosure, the device comprises a container comprised of impermeable material, a mating section in fluid communication with the container, and a one-way valve disposed in the mating section.

### PRIORITY

The present patent application is related to, and claims the priority benefit of, U.S Provisional Patent Application Ser. No. 62/722,851, filed on Aug. 25, 2018, the contents of which are hereby incorporated by reference in their entirety into this disclosure.

### BACKGROUND

Gut micro biota, and more specifically their metabolic products, are closely related to several human biological processes and modulate the processing of many nutrients, drugs, lipids and amino acids. The specific gut metabolites derived from gut micro biota activity are dependent on diet.

The best documented example of a microbial—mammalian metabolic pathway is the choline-derived compound trimethylamine N-oxide (TMAO). TMAO is a volatile gut metabolite generated by gut microbial metabolism. TMAO is produced by intestinal bacteria from trimethylamine, which is derived from phosphatidylcholine choline. Other sources of TMAO are dietary carnitine and lecithin. All are nutrients common in “Western diets.”

TMAO has various physiological effects, including enhanced platelet activation, altering cholesterol metabolism in the intestines, in the liver and in artery walls. The synthesis of TMAO has also been implicated in atherosclerosis progression and other cardiovascular diseases. In fact, TMAO has been identified as an important biomarker of cardiac mortality, myocardial infarction, etc.

Increased levels of TMAO are highly correlated with increased risk of cardiovascular events. TMAO levels are also higher in patients with chronic heart failure than in healthy controls. Moreover, it has been reported that the diet-mediated enhanced synthesis of TMAO increases atherosclerosis progression.

Gut metabolites like TMAO are involved in how a Western diet increases inflammation and progression of different immune-related pathologies such as cardiovascular disease and atherosclerosis.

Because of their demonstrated link to cardiovascular disease, the TMAO produced by gut micro biota have potential as a diagnostic tool and for treatment of disease. It follows that because gut metabolites are involved in many different human biological processes, metabolites other than TMAO can also be tested for and analyzed to diagnose and treat a variety of diseases.

Therefore there is a need for a method and device for collecting gut metabolites like TMAO so they may be tested, measured, analyzed and so forth for diseases. Flatus expelled through the anus is a potential source of metabolites originating in the gut. Furthermore, this source of metabolites is easily accessible, readily available and causes minimal discomfort to the subject/patient. For these reasons it is desirable to provide a novel device that can be used to collect and store flatulence.

### BRIEF SUMMARY

The present disclosure describes a device and method for collecting gut metabolites present in flatus. Although the embodiments below may refer to flatus expelled from the anus, it is understood that the following description is not intended to limit the scope of the present invention device, and it may be used for collection of any expelled fluid or gas from any orifice. In addition, while the specific example of TMAO and cardiovascular disease is used, it is envisioned that many gut metabolites may also be biomarkers for other diseases as well and therefore other gut metabolites may be similarly collected for testing and analyzing.

In an exemplary embodiment of a device for collecting and storing a gaseous sample of the present disclosure, the device comprises a container comprised of impermeable material, a mating section in fluid communication with the container, and a one-way valve disposed in the mating section.

In another exemplary embodiment the device comprises a container comprising an opening and being impermeable except at the opening, a mating section in communication with the opening, and a one-way valve disposed at or near the opening of the container.

The container may be comprised of any suitable material, such as glass, plastic, metal, etc. The device, and more specifically, the container, may be comprised of one or more impermeable materials

In another exemplary embodiment the device comprises a gas binding material disposed inside the container. The gas binding material is configured to bind to the expelled gas and hold the expelled gas in a non gaseous state. The gas binding material may be a solid or liquid. The gas binding material may hold the expelled gas in the same state of matter as the gas binding material or it may hold the expelled gas in a different state of matter as the gas binding material.

In another exemplary embodiment the container and mating section may form a syringe or syringe like device.

In another exemplary embodiment the mating section may have a centimeter diameter and a tip which is flared outward.

In another exemplary embodiment the mating section can be removable for ease of transport.

The mating section comprises a sealing means, wherein the sealing means may be flared tip, a rim or lip, adhesive, suction, etc. In another exemplary embodiment the sealing means may be soft or semi-soft to conform to an uneven surface. In another exemplary embodiment the mating section is flared like a plunger. The mating section may be designed to mate external or internal to the orifice.

The one way valve may be a duckbill valve, umbrella valve, ball valve, dome valve, cross-slit valve, or any other valve known in the art. The one-way valve has a minimal opening and closing pressure. In an exemplary embodiment the minimal opening and closing pressure may be between 1-5 mmHg.

In an exemplary embodiment of a method for collecting and storing a gaseous sample of the present disclosure, the method comprises the steps of: sealing a gas collection device around an orifice, wherein the collection device comprises: a container, a one way valve, and a mating section; expelling gas through the orifice; passing the gas through a one-way valve disposed near the collection device; and holding the expelled gas in the collection device.

In another exemplary embodiment the gaseous sample is flatus. In another exemplary embodiment the orifice is an anus.

In another exemplary embodiment of a method for collecting and storing a gaseous sample of the present disclosure, the collection device further comprises a gas binding material.

The method may further comprise the step of removing the mating section from the gas collection device. The method may further comprise the step of sealing the container.

### BRIEF DESCRIPTION OF THE DRAWINGS

The disclosed embodiments and other features, advantages, and disclosures contained herein, and the matter of attaining them, will become apparent and the present disclosure will be better understood by reference to the following description of various exemplary embodiments of the present disclosure taken in conjunction with the accompanying drawings, wherein:

FIG. 1 shows a sectional lateral view of a gas collection device, according to an exemplary embodiment of the present disclosure;

FIG. 2 shows a sectional lateral view of a gas collection device, according to an alternate embodiment of the present disclosure;

FIG. 3 shows a section lateral view of a first step of collecting gas expelled from an orifice, according to an exemplary method of the present disclosure;

FIG. 4 shows a section lateral view of a second step of collecting gas expelled from an orifice, according to an exemplary method of the present disclosure; and

FIG. 5 shows a sectional lateral view of a gas collection device that has completed gas collection of gas expelled from an orifice, according to an exemplary method of the present disclosure.

An overview of the features, functions and/or configurations of the components depicted in the various figures will now be presented. It should be appreciated that not all of the features of the components of the figures are necessarily described. Some of these non-discussed features, such as various couplers, etc., as well as discussed features are inherent from the figures themselves. Other non-discussed features may be inherent in component geometry and/or configuration.

### DETAILED DESCRIPTION

For the purposes of promoting an understanding of the principles of the present disclosure, reference will now be made to the embodiments illustrated in the drawings, and specific language will be used to describe the same. It will nevertheless be understood that no limitation of the scope of this disclosure is thereby intended.

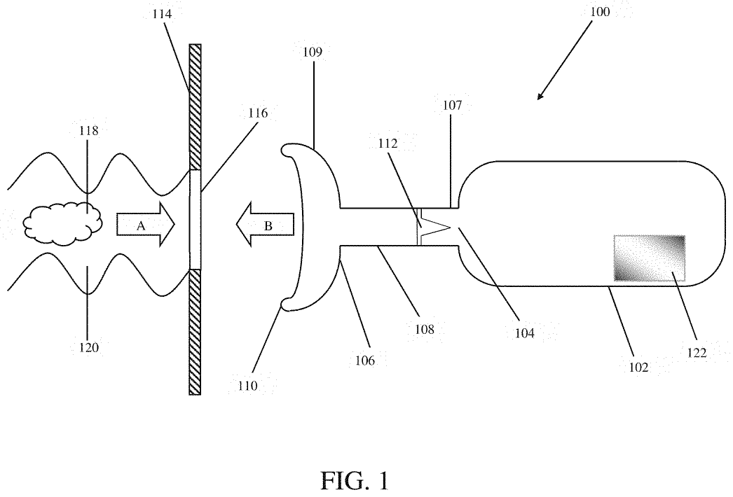

An exemplary device 100 for capturing gaseous emission of the present disclosure is shown in FIG. 1. As shown in FIG. 1, the device 100 comprises a mating section 106 for holding against an orifice 116 and initially receiving expelled gas 118, a one-way valve 112 for controlling the directional flow of the expelled gas 118, a container 102 for accepting and storing the gas 118, and gas binding material 122 for holding the expelled gas 118 in a more controllable form.

The container 102 is made of impermeable material. Any suitable impermeable material may be used such as glass, plastic, metal etc. The container 102 may also take any shape. The container 102 has at least one opening 104 for accepting the expelled gas 118 into the body of the container 102.

In the exemplary embodiment of FIG. 1, the container 102 is made of glass and of a generally cylindrical shape. The embodiment has one opening 104 disposed at one end of the container 102.

As shown in FIG. 1, the device 100 of the present invention also comprises a mating section 106 configured to mate to or seal against and around the orifice 116 expelling gas and also direct the expelled gas 118 into the container 102. The mating section 106 further comprises a sealing means 110 and a first end 107 and a second end 109 and in this embodiment, a neck 108. The mating section 106 comprises the portion of the device 100 extending from the container 102 to the sealing means 110 and is responsible for directing the gas 118 into the container 102. The mating section 106 is also comprised of impermeable material and preferably of the same material as the container 102.

The first end 107 of the mating section 106 is in communication with the opening 104 of the container 102 and extends from the opening 104 of the container 102 towards the second end 109. The mating section 106 may be permanently or removably attached to the opening 104 of the container 102 at the first end 107. The method of attachment may be adhesive, screw-lock, a partial perforation or other weak point, or any other configuration known in the art that establishes an air tight seal such that gas 118 travelling in the direction of arrow A is directed into the container 102.

The sealing means 110 is disposed on the second end 109 and is configured to seal against the orifice 116 that will expel gas. The sealing means 110 may be flared tip, a rim or lip, adhesive, suction source, or other feature that ensures gas 118 expelled in the direction of arrow A will pass through the mating section 106 and into the container 102. The sealing means 110 may also be comprised of an impermeable material. A sealing means 110 that is a rim or lip may be soft or semi-soft to conform to an uneven surface. Further, the mating section 106 can be configured for sealing externally or internally to the body wall 114. The sealing means 110 is responsible for preventing gas 118 leakage during passage into the container 102.

To seal against the orifice 116, the device 100 is advanced against the orifice 116 in the direction of arrow B thereby ensuring that when the orifice 116 opens, gas 118 will travel in the direction of arrow A past the mating section 106, through the one way valve 112, and into the container 102.

In the exemplary embodiment of FIG. 1, mating section 106 has a thin syringe-like neck 108 extending from the opening 104 of the container 102. The neck 108 is at or about a centimeter in diameter and flares outward at the sealing means 110 to a plunger like shape. In this embodiment, the plunger sealing means 110 is flexible, and can be pressed firmly against the body wall 114 and orifice 116. This also creates an additional suction which helps adhere the mating section 106 to the body wall 114. Additionally, when the orifice 116 opens, the additional suction will facilitate the passing of gas 118 from the body canal 120 to the container 102. In the embodiment of FIG. 1, the device 100 takes a syringe like shape.

The one-way valve 112 is disposed in the device 100 such that gas 118 that enters the container 102 must pass through the valve 112 first. This generally means that the valve 112 is positioned somewhere near the midsection of the device 100, or near where the mating section 106 and the container 102 connect. The valve 112 is oriented such that gas 118 may enter the container 102, but not exit. The valve 112 may be a duckbill valve, umbrella valve, ball valve, dome valve, cross-slit valve, or any other valve known in the art. In the embodiment of FIG. 1, the valve 112 is a duckbill valve disposed in the neck 108 of the mating section 106. As illustrated in FIG. 1, the only passage into the container 102 is through the valve 112.

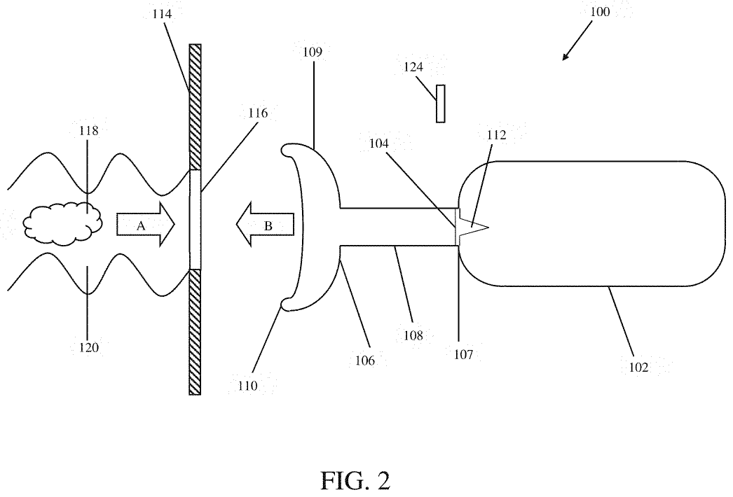

As the valve 112 prevents gas 118 from escaping it may also acts as a cap for the container 102. In the embodiment of FIG. 2, the one-way valve 112 is positioned in the opening 104 of the container 102. This embodiment works best when the mating section 106 is removably attached. In this embodiment, after gas 118 is collected in the container 102, the mating section 106 may be removed for ease of storage and transport. In an alternate embodiment, the one-way valve 112 may be disposed immediately adjacent the opening 104. Generally, in embodiments where the one-way valve 112 is placed outside the mating section 106, the mating section 106 may be removed after gas 118 collection. Where the one way valve 112 acts as the only entrance into the container 102, the mating section 106 may be removed without risk of losing the gas sample 118 in the container 102.

The valve 112 preferably has a minimal opening and closing pressure, between around 1-5 mmHg. After the gas 118 enters the container 102 and the orifice 116 is finished expelling gas, the valve 112 will be forced closed as the pressure in the container 102 is higher than atmospheric pressure. The device 100 can then be removed from the body 114 and processed for storage of the sample 118.

FIG. 1 also shows a solid phase gas binding material 122 disposed in the container 102. The gas binding material 122 has an affinity for the expelled gas 118 and will absorb, bind or otherwise attach to and hold the expelled gas 118. The gas binding material 122 may be either a solid or liquid or a combination and hold the expelled gas 118 in a solid or liquid state. The gas binding material 122 may partially fill, or entirely fill the volume of the container 102, and may take any form such as pellets, grains, fibers, liquids of different viscosities, boiling points, or other characteristics, etc. After collection, the gas 118 may be reconstituted from the gas binding material 122 for further analysis or may also be used in the bound solid or liquid state as is.

If a gas binding material 122 is present, the container 102 may not need to be sealed after gas collection. For example, in one embodiment the device 100 comprises a gas binding material 122 that holds the expelled gas 118 in a solid state. This embodiment further comprises a one-way valve 112 located in a removable mating section 106. After gas collection, the mating section 106 is removed, and therefore the one-way valve 112 is also removed, and the gas sample 118 is stored while bound to the gas binding material 122. This embodiment may also be used with a gas binding material 122 that holds the expelled gas in a liquid state.

For further security a cap 124 and valve 112 may both be used. The cap 124 may screw, snap, or otherwise cover the opening as appropriate to prevent sample loss. For example, the embodiment of FIG. 2 may be further modified with a separate cap 124. After gas 118 is collected and the mating section 106 removed, the separate cap 124 is attached to the opening 104 to further seal the container 102 before storage and transport. A cap 124 could also be used with a non-removable mating section 106. The cap 124 would attach to the second end 109 or the sealing means 110.

The following is an exemplary method of collecting and storing gas 118 expelled from a orifice 116.

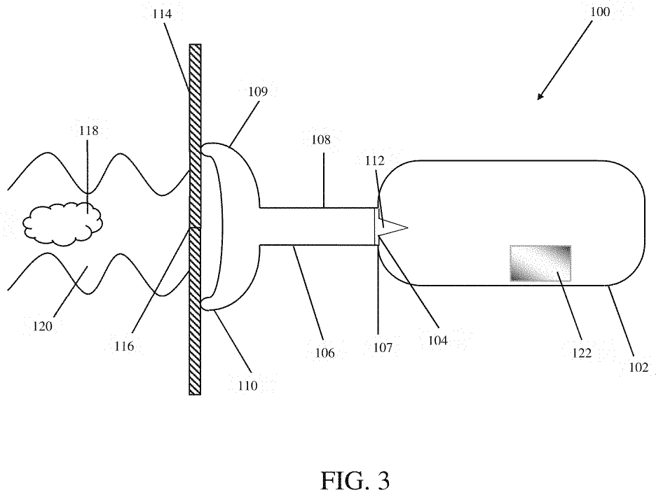

First, the operator shall place an exemplary gas collection device 100 against the orifice 116 expected to expel gas 118 as shown in FIG. 3. The second end 109 of the mating device 100 should be held against the orifice 116 so that the sealing means 110 on the second end 109 of the mating section 106 may seal to the body wall 114 and orifice 116. In the present example, the device 100 will be held against the anus 116 of a patient to collect flatus 118.

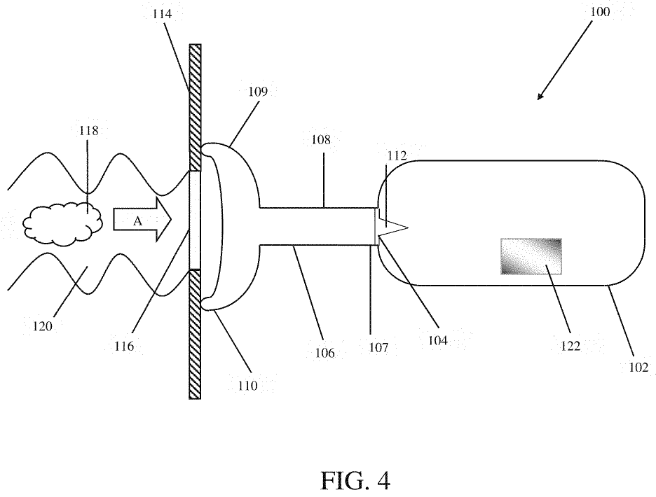

Next, when the device 100 is secured, the patient may open the anus 116 by relaxing the anal sphincter as shown in FIG. 4. The one-way valve 112 has a minimal opening pressure, so the pressure in the body canal 120 from peristalsis will be higher than the opening pressure of the one-way valve 112. Flatus 118 exits the patient through the orifice 116 and enters the second end 109 of the mating section 106. From there it will pass through the one-way valve 112 and into the container 102.

When gas expellation is complete, the one way valve 112 will close and the device 100 may be removed from the anus 116. The one-way valve 112 will stay closed as it has a minimal closing pressure. Furthermore the pressure in the container 102 will be higher than atmospheric pressure preventing the valve 112 from opening.

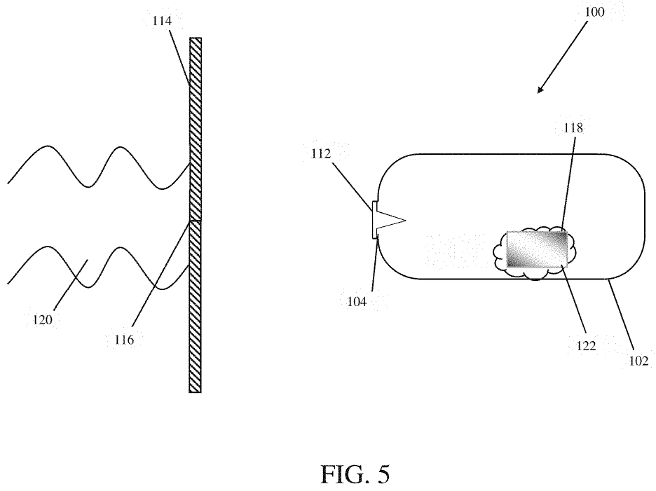

Inside the container 102, a gas binding material 122 is present. The flatus 118 will bind the gas binding material 122 and the mating section 106 is then removed as shown in FIG. 5. Gas collection is complete, and the sample may be additionally capped or immediately transported for analyzing and storing.

While various embodiments of devices for collecting and storing gas expelled from an orifice and methods for the same the same have been described in considerable detail herein, the embodiments are merely offered as non-limiting examples of the disclosure described herein. It will therefore be understood that various changes and modifications may be made, and equivalents may be substituted for elements thereof, without departing from the scope of the present disclosure. The present disclosure is not intended to be exhaustive or limiting with respect to the content thereof.

Further, in describing representative embodiments, the present disclosure may have presented a method and/or a process as a particular sequence of steps. However, to the extent that the method or process does not rely on the particular order of steps set forth therein, the method or process should not be limited to the particular sequence of steps described, as other sequences of steps may be possible. Therefore, the particular order of the steps disclosed herein should not be construed as limitations of the present disclosure. In addition, disclosure directed to a method and/or process should not be limited to the performance of their steps in the order written. Such sequences may be varied and still remain within the scope of the present disclosure.

1. A device for collection and storage of a gaseous sample, the device comprising:
a container;
a mating section in communication with the container; and
a one-way valve disposed in the mating section.

2. The device of claim 1, further comprising:
a gas binding material disposed inside the container.

3. The device of claim 1, wherein the mating section comprises a sealing means.

4. The device of claim 1, wherein the mating section further comprises a first end in communication with the container.

5. The device of claim 4, wherein the mating section comprises a neck with at or about a centimeter diameter and second end which is flared outward.

6. A device for collection and storage of a gaseous sample, the device comprising:
a container comprising an opening;
a mating section in communication with the opening;
a one-way valve disposed at or near the opening of the container.

7. The device of claim 6, wherein the mating section has a first end attached to the opening and a second end flared like a plunger.

8. The device of claim 6, further comprising:
a gas binding material disposed inside the container and configured to bind to the gas sample and hold the gas sample in a non gaseous state.

9. The device of claim 6, wherein the one-way valve has a minimal opening and a minimal closing pressure.

10. The device of claim 9, wherein the minimal opening and minimal closing pressure is at or between 1-5 mmHg

11. The device of claim 6, wherein the mating section is removably attached to the container.

12. The device of claim 6, wherein the mating section comprises a sealing means and said sealing means may be soft or semi-soft.

13. A method for collecting and storing a gaseous sample expelled from an orifice, the method comprising the steps of:
sealing a gas collection device around an orifice, wherein the collection device comprises:
a container, a one way valve, and a mating section;
expelling gas through the orifice;
passing the gas through a one-way valve disposed near the collection device; and
holding the expelled gas in the collection device.

14. The method of claim 13, wherein the gaseous sample is flatus.

15. The method of claim 14, wherein the orifice is an anus.

16. The method of claim 13, wherein the gas collection device further comprises a gas binding material.

17. The method of claim 13, further comprising the step of removing the mating section from the gas collection device.

18. The method of claim 17, further comprising the step of sealing the container.

19. The method of claim 16, further comprising the step of removing the mating section from the gas collection device.

20. The method of claim 19, further comprising the step of sealing the container.
